(19) United States
(12) Patent Application Publication
(10) Pub. No.: US 2009/0221879 A1
(43) Pub. Date: Sep. 3, 2009

(54) Minimally Invasive Retractor Having Separable Blades

(75) Inventor: Josef Gorek, Ross, CA (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/396,150

(22) Filed: Mar. 2, 2009

Related U.S. Application Data
(60) Provisional application No. 61/032,188, filed on Feb. 28, 2008.

(51) Int. Cl. A61B 1/32; A61B 17/88

(52) U.S. Cl. 600/214; 606/279

(57) ABSTRACT

A device, system and method for orthopedic spine surgery using a screw-based retractor are disclosed herein and allows for access to the spine through a minimally or less invasive approach. The retractor device is designed to be coupled to a pedicle screw and then to have opposed arms of the retractor spread apart to open the wound proximally. The retractor is removed by pulling it out of the wound whereby the retractor is separated along an asymmetric stress riser to pass over the pedicle screw head. The retractor is intended to be made of a flexible metal material, sterile packaged and disposable after one use. A system and method for using the retractor and performing a minimally invasive spine surgical procedure are also disclosed.

### CROSS-REFERENCE TO RELATED APPLICATIONS

The present application claims priority to, and the benefit of, U.S. Provisional Application Ser. No. 61/032,188 filed Feb. 28, 2008, the entire contents of which are hereby incorporated by reference.

### BACKGROUND

1. Technical Field

The present disclosure relates generally to orthopaedic spine surgery and in particular to a minimally invasive retractor and methods for use in a minimally invasive surgical procedure.

2. Background of the Technology

There has been considerable development of retractors and retractor systems that are adapted for use in less invasive procedures. Many of the recent developments are based on traditional types of surgical retractors for open procedures, predominantly table-mounted devices of various designs. These devices tend to be cumbersome and are not well adapted for use in small incisions. Standard hand-held surgical retractors are well known in the prior art and can be modified to fit the contours of these small incisions, but they require manual manipulation to maintain a desired placement, thereby occupying one hand of the physician or requiring another person to assist the physician during the procedure. Typical retractors are also positioned into the soft tissue and are levered back to hold the wound open, frequently requiring re-positioning if they dislodge, obstruct the physician's view, or interfere with access to the surgical site.

In recent years, minimally invasive surgical approaches have been applied to orthopedic surgery and more recently to spine surgery, such as instrumented fusions involving one or more vertebral bodies. Unlike minimally invasive procedures such as arthroscopic knee surgery or gallbladder surgery where the affected area is contained within a small region of the body, spinal fusion surgery typically encompasses a considerably larger region of the patient's body. In addition, arthroscopic surgery and laparoscopic surgery permit the introduction of fluid (i.e. liquid or gas) for distending tissue and creating working space for the surgeon. Surgery on the spine does not involve a capsule or space that can be so distended, instead involving multiple layers of soft tissue, bone, ligaments, and nerves. For these reasons, the idea of performing a minimally invasive procedure on the spine has only recently been approached.

By way of example, in a typical spine fusion at least two vertebral bodies are rigidly connected using screws implanted into the respective vertebral bodies with a solid metal rod spanning the distance between the screws. This procedure is not generally conducive to a minimally invasive approach. The insertion of pedicle or facet screws is relatively straightforward and can be accomplished through a minimal incision. The difficulty arises upon the introduction of a length of rod into a very small incision with extremely limited access and visibility. A single level fusion may require a 30-40 mm rod to be introduced into a 1 cm incision and a multilevel fusion may require a rod several inches long to fit into a 1 cm incision. For this reason, it is important that the minimal incision be maintained in an open and accessible condition (i.e. as wide as practicable) for introduction of the rod.

Minimally invasive surgery offers significant advantages over conventional open surgery. First, the skin incision and subsequent scar are significantly smaller. By using more than one small incision rather than one large incision, the need for extensive tissue and muscle retraction may be greatly reduced. This leads to significantly reduced post-operative pain, a shorter hospital stay, and a faster overall recovery.

Most spine implant procedures are open procedures, and while many manufacturers advertise a minimally invasive method, the procedure is typically not recommended for fusions and focuses on more common and accepted minimally invasive spine procedures such as kyphoplasty, vertebroplasty, and discectomy.

Medtronic Sofamor Danek's SEXTANT® is a minimally invasive device used for screw and rod insertion. Its shortcomings lie with how complicated the system is to use and the requirement for an additional incision for rod introduction. This system also requires that the guidance devices be rigidly fixed to the pedicle screw head in order to maintain instrument alignment and to prevent cross-threading of the setscrew. For these reasons, the surgeon cannot access the surrounding anatomy for complete preparation of the field. Nor does SEXTANT® allow for any variation in the procedure, if need be.

Depuy Spine's VIPER™ system is another minimally invasive implant and technique recommended for one or two level spine fusions. This system is less complicated than the SEXTANT® only requiring two incisions for a unilateral, one-level fusion, but it is limited in the same way as the SEXTANT® because it also requires the instrumentation to be rigidly fixed to the pedicle screw.

Spinal Concept's PATHFINDER® and NuVasive's SPHERX® spinal system (as disclosed in U.S. Pat. No. 6,802,844), are marketed as “minimally disruptive” spine fusion implants and procedures. While they have advantages over a general “open” procedure, they do not provide all of the advantages of a truly minimally invasive approach. Their characterization as “minimally open” procedures is a result of the inherent difficulty of introducing a rod in a minimally invasive spinal procedure. In order to introduce a rod long enough to accomplish a single level fusion, these systems describe an incision long enough to accept such a rod, thereby undermining the advantages of a minimally invasive approach.

The problem of rod introduction warrants further discussion as it is the central problem in minimally invasive spinal fusions. The systems currently on the market address this issue by adding another incision, using a larger incision, or avoiding fusions greater than one level.

In order to be truly minimally invasive, a spine fusion procedure should have a minimum number of small incisions and not require significant tissue and/or muscle retraction. Furthermore, an improved approach should encompass as many variations and applications as possible thereby allowing the surgeon to adjust the procedure to accommodate the anatomy and surgical needs of the patient as presented. For instance, spinal fusions should not be limited to just one or two levels.

Therefore, a continuing need exists for an improved device, an improved system, and an improved method for performing minimally invasive spine surgery.

### SUMMARY

The present disclosure relates to a device, a system, and a method for a screw-based retractor used in performing minimally invasive spine surgery. The retractor is removably attached to a pedicle bone screw that is used to guide the retractor into place and act as a point of fixation with respect to the patient. Multiple retractors may be used in conjunction with a single screw to allow retraction in multiple directions and multiple retractors may be used with multiple screws, respectively, during a single spine procedure. The retractor may be manufactured for a single use or can be sterilized and reused. Finally, the retractor may also act as a guide that will aid in the insertion of instruments and implants.

In its nominal position, the retractor extends longitudinally from the screw in a generally cylindrical shape with at least one retracting blade. Instrument holes are located perpendicular to the long axis of each retracting blade whereby a standard surgical instrument, such as a Gelpi retractor, can be used to separate the blades to retract the skin and soft tissue and maintain the field of view as well as a site for performing surgical procedures. Yet, where the retractor is connected to the pedicle screw the retractor maintains a substantially circular cross-section. Since the retractor is not permanently fixed but is removably attached to the pedicle screw, it is free to have polyaxial rotation allowing the surgeon greater wound access and freedom to operate. Furthermore, polyaxial rotation allows the retractor to expand medial-laterally as well as cephalad-caudally and any combination thereof. This freedom of movement proximally and non-rigid attachment distally decreases the need for retractor re-positioning during a procedure. Proximal stabilization of the retractor is possible when it is used in conjunction with either a free standing or table-mounted retractor.

The minimally invasive retractor can be designed to flex proximal or distal to the pedicle screw head. In one embodiment, the retractor has a “living hinge” incorporated into the retractor's blade design. In a further embodiment, the minimally invasive retractor has a pair of living hinges. It is contemplated that the minimally invasive retractor may have a pair of true hinges.

As viewed along its longitudinal axis, a cross-section of the minimally invasive retractor has a generally circular configuration and provides additional stiffness. The geometry of the minimally invasive retractor provides sufficient stiffness for maintaining the opening at the surgical site.

Minimally invasive retractors having combinations of a living hinge and/or a true hinge may include at least one window that is aligned with the pedicle screw saddle and allows the insertion of instruments into the surgical site.

The distal tip of the minimally invasive retractor is bullet shaped to aid in insertion through the soft tissue to where it will seat against the pedicle. The distal tip will also have one or more stress risers formed therein to aid in removing the retractor. Upon completion of the procedure, the retractor can be pulled straight out of the wound and the distal tip will separate along the stress risers to pass over the screw and rod assembly. Advantageously, by positioning the distal tip of the retractor around the head of the screw adjacent the bone, the retractor retracts soft tissue from a point below the head of the screw, creating excellent visibility of the screw and surrounding tissue.

### BRIEF DESCRIPTION OF THE DRAWINGS

Embodiments of the presently disclosed minimally invasive retractor are described herein with reference to the accompanying drawings, wherein:

FIG. 1 is a perspective view of a minimally invasive retractor according to an embodiment of the present disclosure;

FIG. 2 is a bottom view of the minimally invasive retractor of FIG. 1;

FIG. 3 is a side view of a minimally invasive retractor according to another embodiment of the present disclosure;

FIG. 3A is an enlarged detail view from FIG. 3 of the minimally invasive retractor;

FIG. 3B is a top view of the minimally invasive retractor of FIG. 1;

FIG. 4 is a perspective view of a minimally invasive retractor and screw assembly including the minimally invasive retractor of FIG. 3;

FIG. 5 is a side view of the minimally invasive retractor and screw assembly of FIG. 4;

FIG. 6 is an enlarged detail view from FIG. 5 of the minimally invasive retractor; and

FIG. 7 is a front cross-sectional view of a vertebral body with a pair of minimally invasive retractors attached using screws with the blades in their initial position and rods positioned in the passages of the minimally invasive retractors.

### DETAILED DESCRIPTION

Embodiments of the presently disclosed minimally invasive retraction device will now be described in detail with reference to the drawings wherein like reference numerals identify similar or identical elements. In the drawings and in the description which follows, the term “proximal”, as is traditional, will refer to the end of the minimally invasive retraction device which is closest to the operator while the term “distal” will refer to the end of the device which is furthest from the operator.

The present disclosure relates to a device, a system, and a method for a screw-based retractor used in performing minimally invasive surgery. Such a device, system, and method is disclosed in U.S. patent application Ser. No. 11/528,223 filed on Sep. 26, 2006 (U.S. Patent Application Publication No. 2007/0106123), the entire contents of which are incorporated herein by reference.

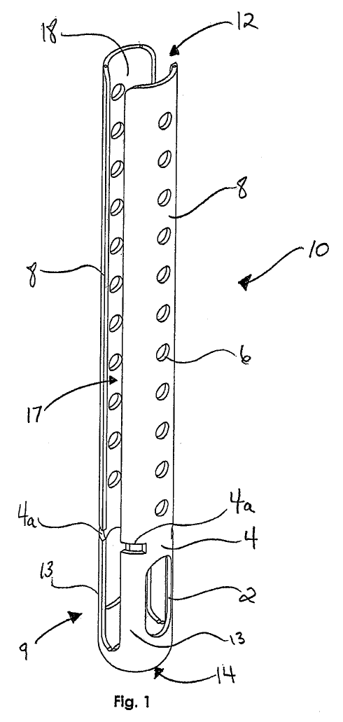

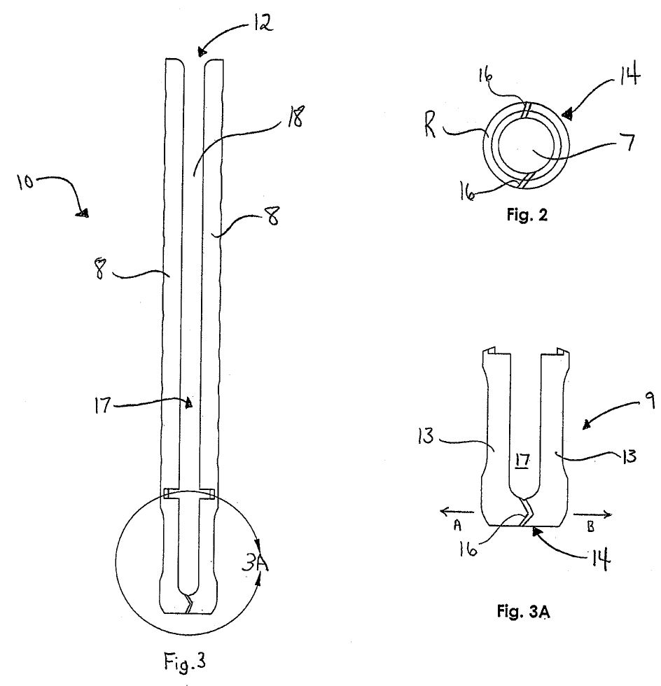

Referring intially to FIGS. 1, 2, 3, 3A, and 3B, an embodiment of the presently disclosed minimally invasive retractor is illustrated and generally designated as 10. Retractor 10 includes an open proximal end 12 and a distal end 14. In addition, retractor 10 includes a pair of retractor blades 8 having a plurality of instrument holes 6 disposed on each of retractor blades 8. Instrument holes 6 are configured and dimensioned to cooperate with different surgical instruments (e.g., a Gelpi retractor). A distal region 9 of retractor 10 includes an opening 7 (FIG. 2), optionally at least one slot or window 2, and a pair of arms 13 extending from distal end 14 to a flexible region or living hinge 4. Window 2 is sized and configured to receive instruments therethrough and/or permit visual inspection. Each retractor blade 8 is attached to living hinge 4 to define a substantially continuous elongate member. A pair of recesses 4a are formed between retractor blade 8 and arm 13 to define living hinge 4.

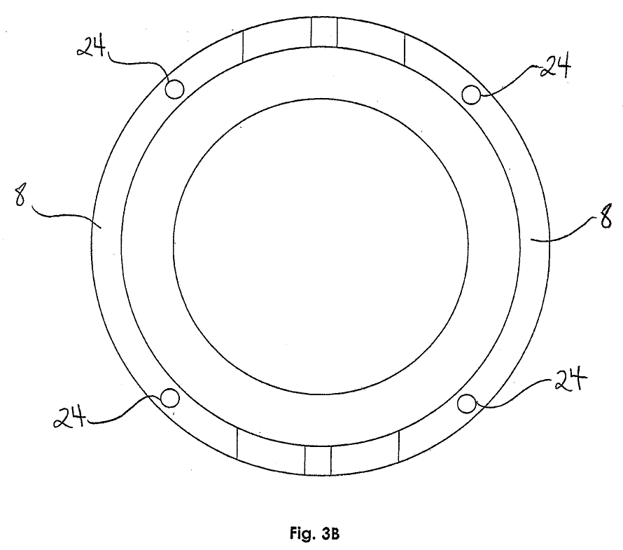

Distal end 14 further includes at least one disengagement region R (FIG. 2) defined by at least one stress riser 16 extending proximally from opening 7 (FIG. 2). Alternatively, stress riser 16 may originate at window 2 and extend distally towards opening 7. It is contemplated that other arrangements of relief structures may be used to define disengagement region R and these may exist between opening 7 and window 2. Each stress riser 16 is a weakened portion of distal end 14. It may be a score in the material, a perforated region in the material, or another structural arrangement acting in a sacrificial capacity to allow disengagement region R to be radially separated away from the centerline of retractor 10 in response to applied forces as indicated by directional arrows A and B (FIG. 3A). In addition, distal end 14 has a generally convex outer surface that facilitates insertion of retractor 10 through layers of body tissue.

In the illustrated embodiment, stress risers 16 are formed on distal end 14 in an asymmetrical configuration. More particularly, as stress risers 16 extend proximally from opening 7, they are biased in the direction of directional arrow B (e.g., medially). It is contemplated that as stress risers 16 extend proximally from opening 7, they may be biased in the direction of directional arrow A (e.g., laterally).

Retractor blades 8 and arms 13 are generally arcuate structures that cooperate to define a substantially circular configuration for retractor 10. Each retractor blade 8 and each arm 13 have an arcuate configuration that is less than about 180° and are radially spaced apart to define a continuous slot 17 along a substantial portion of retractor 10. In addition, each retractor blade 8 and its corresponding arm 13 define a passage 18 that also extends substantially the entire length of retractor 10. Passage 18 is expandable, as will be discussed in detail hereinafter, for receiving a rod 3 (FIG. 7) therein. Retractor blades 8 and arms 13 define a substantially circular ring shape, thereby providing sufficient stiffness (i.e. rigidity) such that retractor blades 8 and arms 13 resist bending from the counter forces of the retracted tissues.

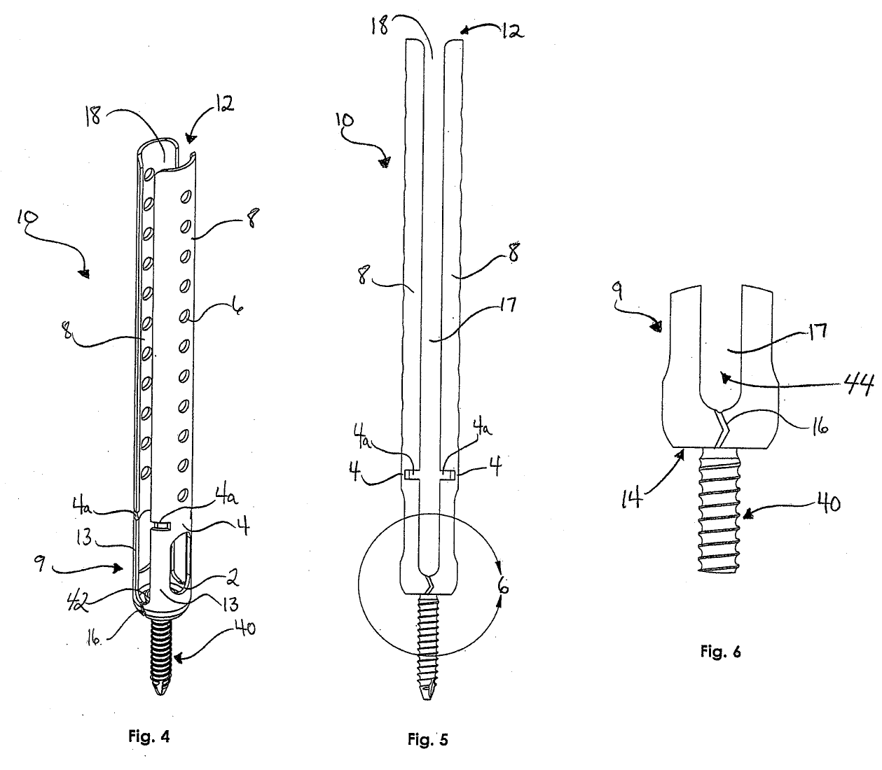

Opening 7 is located at distal end 14 of retractor 10 and is sized for receiving the shank of a threaded screw 40 (FIGS. 4-6) therethrough, but inhibiting passage of a head 42 (FIG. 4) of screw 40 so as to support screw 40 at distal end 14 of retractor 10. The interior surface of distal end 14 has a generally concave spherical geometry that is adapted to mate with head 42 of pedicle screw 40.

Retractor 10 is formed from a suitable biocompatible material having the desired physical properties. That is, retractor 10 is formed of a biocompatible, sterilizable material in a suitable configuration and thickness so as to be sufficiently rigid to be held on the screw when desired during insertion and a surgical procedure and to provide retraction of tissue, and yet is sufficiently bendable to be spread apart to provide retraction and to be forcibly removed from the screw as necessary and appropriate. Examples of suitable biocompatible materials include polypropylene, polyethylene, polycarbonate, silicone, and polyetheretherketone. It is contemplated that retractor 10 may be formed from metal. Any maleable, bendable, flexible, or otherwise formable metal known in the art may be used, such as titanium, titanium alloy, surgical stainless steel, shape memory alloy, etc. A non-conductive coating may be applied to the surface of retractor 10 to allow for electrical stimulation of threaded screw 40 (FIGS. 4-6) without shunting of current through retractor 10. Any suitable non-conductive dielectric material known in the art may be applied to retractor 10 to achieve this purpose.

Referring now to FIG. 3B, one or more channels or tubes 24 are defined through the longitudinal cross-section of retractor blades 8. Tubes 24 may be defined along the entire length of retractor blades 8 and are adapted to accommodate optical fiber (not shown) therethrough. The optical fiber is in optical communication with any suitable energy source known in the art (not explicitly shown) and utilized to illuminate the length of retractor 10, or any particular portion thereof, from proximal end 12 along continuous slot 17 to distal end 14. Additionally or alternatively, channels or tubes 24 may be formed from an optically transmissive material, as is known in the art.

Retractor blade 8 is bendable away from the centerline of retractor 10 in response to applied forces, wherein retractor blade 8 bends at living hinge 4 and/or possibly below the living hinge. Bending retractor blade 8 away from the centerline (i.e. radially outwards) creates a larger opening through retractor 10 and also acts to retract the surrounding tissue at the selected surgical site. Installation and use of retractor 10 in surgical procedures will be discussed in detail hereinafter.

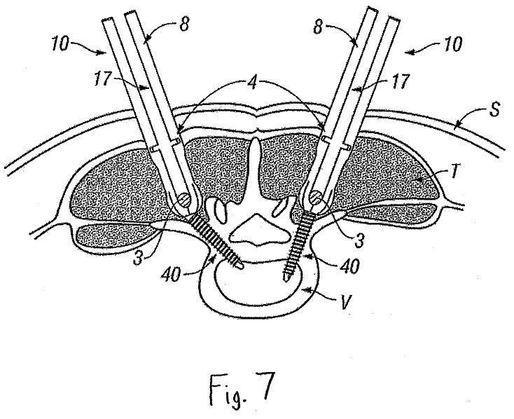

In FIGS. 4-6, retractor 10 is illustrated in an assembled condition with a pedicle screw 40. Pedicle screw 40 extends through opening 7 (FIG. 6) such that threads of pedicle screw 40 extend beyond distal end 14 (FIG. 6) for insertion into a target site in a bone (e.g. a vertebral body). As shown in the figures, when pedicle screw 40 is inserted in retractor 10, the head of pedicle screw 42 (FIG. 4) mates with the interior geometry of distal end 14. As shown, rod receiving passage 44 (FIG. 6) aligns with opening 17 between retractor blades 8 facilitating the insertion of rod 3 (FIG. 7) into screw head 42. In addition, pedicle screw 40 is pivotable about the longitudinal axis of retractor 10 allowing retractor 10 to be attached in a first angular orientation with respect to the vertebral body, but pivotable about pedicle screw 40 increasing the amount of tissue that may be retracted using retractor 10.

It is contemplated that retractor 10 may be formed of a bendable resilient material such that when external spreading forces (i.e., from a Gelpi retractor or the physician's hands) are removed, the retractor blades may return towards their initial position (e.g., substantially parallel to the centerline). It is also contemplated that retractor 10 may be formed of a bendable non-resilient material such that when the external spreading forces are removed, the retractor blades resist returning to their initial position and remain in the retracted position.

A method for use of the presently disclosed system will now be described with reference to FIG. 7. Retractor 10 is assembled with pedicle screw 40 as shown in FIG. 7. The assembled apparatus is inserted into an incision through the patient's skin S and muscle/fat tissue T such that pedicle screw 40 is subsequently threaded into a vertebral body V. Once the desired number of retractors 10 are affixed to vertebral body V, retractor blades 8 are spread apart to retract skin S and tissue T to create a retracted area at the target site. Rod 3 is inserted in passage 18 when passage 18 is in an expanded state (i.e., tissue has been retracted). Additionally, rod 3 is repositioned through passage 18 and subcutaneously such that is may be secured to fastening regions of pedicle screws in adjacent vertebral bodies. Once the screw-rod construct is complete, retractor 10 is removed from the patient using, for example, a retractor extracting tool. Such a retractor extracting tool is described in U.S. Patent Application Serial No. 11/528,223 (referenced hereinabove). A physician pulls retractor 10 proximally, such that disengagement regions R (FIG. 2) separate, preferably along stress risers 16, radially away from the centerline of retractor 10, as indicated by directional arrows A and B (FIG. 3A). As such, retractor 10 is separated from pedicle screw 40 without imparting significant downward or rotational forces against the patient's body. Retractor 10 may now be removed from the patient and this process may be repeated for each installed retractor.

As will be appreciated, the pedicle screw may be cannulated such that it may be translated along a guide wire, thereby facilitating insertion of the pedicle screw and the minimally invasive retractor into the work site. In addition, it is contemplated that conventional insertion tools or those disclosed in U.S. Provisional Patent Application Ser. No. 60/925,056, filed on Apr. 17, 2007, the entire contents of which are hereby incorporated by reference may be used in conjunction with the presently disclosed minimally invasive retractors and pedicle screws.

It is contemplated that the retractor may be utilized in, but not limited to, a method whereby an initial incision is made in the skin of approximately 10-15 mm in length. Surgeon preference will dictate the need for one or more stages of dilators to aid in expanding the wound before introducing one or more retractors in combination with pedicle screws. Normal surgical techniques may be used to close the incision(s).

It is also contemplated that the retractor may be manufactured from medical grade metal or composites of metal. A metallic part utilizes such materials as, but not limited to, aluminum, stainless steel, nickel-titanium, titanium and alloys of the foregoing. In addition, the parts may have a reflective or non-reflective coating to aid in increasing visibility in the wound and may have an artificial lighting feature.

As with any surgical instrument and implant, the retractors must have the ability to be sterilized using known materials and techniques. Parts may be sterile packed by the manufacturer or sterilized on site by the user. Sterile packed parts may be individually packed or packed in any desirable quantity. For example, a sterile package may contain one or a plurality of retractors in a sterile enclosure. Alternatively, such a sterile surgical kit may also include one or a plurality of bone biopsy needles guide wires, sterile cannulated scalpels, dilators, rods, or other surgical instruments.

It will be understood that various modifications may be made to the embodiments of the presently disclosed retraction system. Therefore, the above description should not be construed as limiting, but merely as exemplifications of embodiments. Those skilled in the art will envision other modifications within the scope and spirit of the present disclosure.

For example, while the foregoing description has focused on spine surgery, it is contemplated that the retractors and methods described herein may find use in other orthopedic surgery applications, such as trauma surgery. Thus, where it is desired to insert a screw or pin into bone in a minimally invasive manner, or otherwise to access a surgical target site over a guidewire, the dilator, scalpel and retractors (or some of them) of the present disclosure may be used, with or without a bone screw.

The blades may be made of a light transmitting material. The retractor may include a light guide system. The light guide system has an input adapter to receive light from a light source and one or more light emitting surfaces to illuminate the surgical field.

Further still, it will be appreciated that the pedicle screw may be cannulated such that it may be translated along a guide wire, thereby facilitating insertion of the pedicle screw and retractor. In addition, it is contemplated that conventional insertion tools or those disclosed in U.S. patent application Ser. No. 12/104,653, filed on Apr. 17, 2008 (U.S. Patent Application Publication No. 2008/0262318), the entire contents of which are hereby incorporated by reference may be used in conjunction with the presently disclosed retractor and pedicle screws.

What is claimed is:

1. A surgical retractor comprising:
a pair of opposed elongate members;
a coupling region disposed at one end of the surgical retractor, the coupling region having an opening located at a distal end thereof and at least one disengagement region comprising at least one stress riser; and
at least one flexible joint coupling each of the elongate members to the coupling region.

2. The surgical retractor of claim 1, wherein the flexible joint is a living hinge.

3. The surgical retractor of claim 1, wherein the at least one stress riser extends proximally from the opening along the disengagement region.

4. The surgical retractor of claim 1, wherein the coupling region includes at least one widow defined therethrough, the at least one window configured to receive instruments therethrough.

5. The surgical retractor of claim 4, wherein the at least one stress riser extends between the at least one window and the opening.

6. The surgical retractor of claim 1, wherein the surgical retractor is made from a malleable metal.

7. The surgical retractor of claim 1, wherein the surgical retractor is formed from a material selected from the group consisting of: titanium, titanium alloy, surgical stainless steel, and shape memory alloy.

8. The surgical retractor of claim 1, wherein the surgical retractor is formed from a material selected from the group consisting of: polypropylene, polyethylene, polycarbonate, silicone, and polyetheretherketone.

9. The surgical retractor of claim 1, wherein the stress riser is asymmetric with respect to a longtidunial axis of the retractor.

10. The surgical retractor of claim 1, further including a bone screw extending from a distal end of the surgical retractor.

11. A surgical retractor, comprising:
a pair of opposed elongate members;
a coupling region disposed at one end of the surgical retractor and operably coupled to the pair of opposed elongate members, the coupling region having an opening located at a distal end thereof and at least one disengagement region including at least one stress riser extending proximally from the opening along the coupling region; and
at least one living hinge connecting each of the opposing elongate members to the coupling region.

12. The surgical retractor of claim 11, wherein the coupling region includes at least one widow defined therethrough, the at least one window configured to receive instruments therethrough.

13. The surgical retractor of claim 12, wherein the at least one stress riser extends between the at least one window and the opening.

14. The surgical retractor of claim 11, wherein the surgical retractor is made from a malleable metal.

15. The surgical retractor of claim 11, wherein the at least one living hinge is defined by a pair of opposing recesses formed between the pair of opposed elongate members and the coupling region.

16. A method of retracting tissue, comprising the steps of:
inserting a surgical retractor into an incision in a patient's skin, the surgical retractor having a coupling region disposed at one end thereof, the coupling region having an opening located at a distal end thereof and at least one stress riser extending proximally from the opening;
moving opposed blades of the surgical retractor away from a centerline thereof, thereby enlarging the incision;
moving the surgical retractor proximally such that the coupling region separates along the at least one stress riser away from the centerline of the surgical retractor;
separating the opposed blades of the surgical retractor; and
removing the separated blades of the surgical retractor.

17. A method of performing spine surgery comprising the steps of:
a) providing at least two retractor assemblies, each retractor assembly including a pair of opposing elongate members and at least one stress riser located at a distal end thereof, the pair of opposing elongate members being flexibly and releasably coupled to a pedicle screw;
b) securing the first screw to a portion of a first vertebral body;
c) retracting tissue using the at least one elongate member of the first retractor;
d) securing the second screw to a portion of a second vertebral body;
e) retracting tissue using the pair of opposing elongate members of the second retractor;
f) inserting a rod between the first and second screws;
g) securing the rod to the first and second screws;
h) moving the surgical retractor proximally such that the pair of opposing elongate members separates along the at least one stress riser away from the centerline of the surgical retractor; and
h) removing the elongate members from the pedicle screws.
